(19) United States
(12) Patent Application Publication
(10) Pub. No.: US 2025/0387210 A1
(43) Pub. Date: Dec. 25, 2025

(54) METHOD FOR CLEANING TEETH AND CLEANING SYSTEM

(71) Applicant: epitome GmbH, Wien (AT)

(72) Inventors: Branislav BUKOVINSKY, Pezinok (SK); Bernhard TRINNES, Langenlois (AT); Christian FEICHTER, Wien (AT)

(21) Appl. No.: 18/877,372

(22) PCT Filed: Jun. 20, 2023
(86) PCT No.: PCT/EP2023/066685

§ 371 Date: Dec. 20, 2024

(30) Foreign Application Priority Data
Jun. 21, 2022 (DE) 10 2022 115 396.7

(51) Int. Cl. A61C 17/02; A61C 19/04

(52) U.S. Cl. CPC A61C 17/0217; A61C 19/04

(57) ABSTRACT

A method for cleaning teeth using a tooth-cleaning device by removing tooth stains uses the following models: a tooth model, which describes the tooth structure of a user, a cleaning model of the particular user, which describes the tooth model by means of a state of staining, an expectation cleaning model, which describes the tooth model by means of an expected state of cleaning, an actual cleaning model, which is measured by means of corresponding sensors, and a cleaning deviation, which is determined by a comparison of the expected cleaning model with the actual cleaning model. The method comprises the steps of: a) determining the cleaning model and the expectation cleaning model; b) performing a cleaning process as defined by the cleaning model; c) determining the actual cleaning model; d) deriving the cleaning deviation from the actual cleaning model and the expectation cleaning model; e) using the actual cleaning model as new cleaning model, determining the expectation cleaning model on the basis of the new cleaning model, and adapting cleaning instructions on the basis of the cleaning deviation; and f) repeating steps b) to e) until a predetermined termination criterion has been reached.

The present invention relates to a method for cleaning teeth with a cleaning device and a cleaning system.

Like daily washing, tooth brushing is an essential part of cleaning the human body. Nevertheless, the technique has not changed significantly over the last few centuries.

Most users use simple, analogue manual toothbrushes or electrically operated toothbrushes, which are designed like manual toothbrushes but have a motorized, movable head so that a vibration of the head is generated.

There is no difference between the two systems in terms of the basic method of brushing teeth, however.

There are known U-shaped toothbrushes that have a U-shaped design and can clean all of the teeth simultaneously. This process is also known as 3D cleaning.

US 2020/0179089 A1 discloses an oral hygiene monitoring system that monitors the movement and orientation of oral hygiene devices, such as a toothbrush, during use. This is done by means of one or more cameras that monitor the movement of the toothbrush from outside the body of the person cleaning his teeth. A camera can also be positioned on the oral hygiene device itself and can be used to detect the tooth quality.

US 2020/0201266 A1 discloses a household cleaning appliance. This cleaning appliance can be embodied for a wide variety of applications such as cleaning the floor of a building, shaving a human body, or cleaning teeth. This cleaning appliance can have a neural network with which it is possible to determine different properties of an image of the object to be cleaned, for example the color of teeth, in order to be able to influence the cleaning process.

US 2020/0121428 A1 shows a tooth cleaning device that has a U-shaped and curved rail so that a row of teeth can be enclosed with it. Tooth cleaning elements such as bristles are provided on the inside of the rail so that all of the teeth of a row of teeth can be cleaned simultaneously with this device. This tooth cleaning device can be embodied in such a way that individual cleaning plans are executed for rows of teeth of a specific person.

US 2021/0393026 A1 describes an oral hygiene system that is a type of intelligent toothbrush, which has an optical sensor to optically scan the inside of the mouth. The sensor data can be analyzed with a machine learning system such as a neural network.

US 2012/0171657 A1 discloses a cleaning system such as an electric toothbrush, which is connected to a display device on which the user is shown information and instructions on how to use the cleaning device.

The object of the invention is to clean teeth thoroughly and quickly.

One or more objects are attained by the subject of the independent claim.

Advantageous embodiments and preferred embodiments are the subjects of the dependent claims.

A method for cleaning teeth with a tooth cleaning device for removing plaque uses the following models:
a dental model that describes the tooth structure of a user,
a cleaning model of the respective user, which describes the dental model with a plaque condition, wherein the cleaning model is used as a basis for establishing cleaning instructions for a tooth cleaning process,
an expected cleaning model, which describes the dental model with an expected cleaning state that should be achieved after performing a specific cleaning procedure as defined by the cleaning model,
an actual cleaning model that is actually achieved after performing the particular cleaning operation as defined by the cleaning model, wherein the actual cleaning model is measured with corresponding sensors,
a cleaning deviation, which is determined by comparing the expected cleaning model to the actual cleaning model.
a) determination of the cleaning model and the expectation-cleaning model,
b) execution of a cleaning process as defined by the cleaning model,
c) determination of the actual cleaning model,
d) derivation of the cleaning deviation from the actual cleaning model and the expected cleaning model,
e) use of the actual cleaning model as the new cleaning model, determination of the expected cleaning model based on the new cleaning model, and adjustment of the cleaning rules based on the cleaning deviation,
f) repetition of steps b) to e) until a predetermined termination criterion is reached.

Many users brush their teeth regularly. However, only very few brush their teeth consciously or focus their attention on the brushing process. People often look at their smartphone, watch TV, or perform minor tasks while brushing. Some people brush their teeth in the shower. The fact that a user does not focus on the brushing process makes it comparatively difficult to carry out the actions recommended by the dentist for brushing teeth. This includes, for example, thoroughly brushing the necks of the molars or the insides of the lower incisors. Users do not usually focus on the amount of plaque on their teeth, but instead carry out their routine brushing behavior in the same way every time. Variations in this way of doing things arise at most through external factors, such as stress, fatigue, or distraction. For example, users who are under time pressure will clean their teeth less thoroughly than users who are fully focused on brushing their teeth. As a result, teeth are often not cleaned sufficiently, increasing the likelihood of tooth decay and other dental diseases.

The advantage of the present invention is that the user's plaque condition is determined independently of the user and that based on the plaque condition, a cleaning is then carried out that is dependent on this plaque condition. A user then no longer needs to concentrate on the actual cleaning process and still receives a thorough tooth cleaning.

The individual position of a user's teeth, as well as the user's behavior during the cleaning process, the frequency of the cleaning processes, and diet all influence the quality of the cleaning process with a cleaning device. Recording all of these parameters and taking them into account in the instructions for the cleaning processes can be very time-consuming or even impossible. For example, if the user is a smoker, he or she would have to record the time of each cigarette smoked in order to take cigarette staining into account. As described above, certain tooth positions can also minimize the cleaning success of a preset cleaning device.

To solve these problems, the success of the cleaning process is checked at regular intervals in the control loop and compared to an expected cleaning success, as illustrated above. This means that with each new cleaning model, the cleaning model is continuously adjusted to the actual conditions and the user's behavior. As a result, the cleaning model may be very similar for all users at the beginning of the control loop, but the further the control loop progresses, i.e. the more cycles it executes, the more individual the user's cleaning model is compared to other users.

The cleaning device can be embodied in such a way that a U-shaped section of the cleaning device is inserted into a user's oral cavity. The U-shaped section of the cleaning device has a nozzle arrangement and is placed over the teeth. A nozzle is arranged above each tooth surface. The nozzles spray a cleaning fluid onto the teeth and in so doing, remove the plaque. The cleaning instructions include parameters for operating the cleaning device such as the cleaning duration, pressure of the fluid jet, intensity, and/or direction.

The cleaning device can also be embodied with an automatic or semi-automatic brushing device as an alternative or in combination with the nozzle arrangement. The brushing device can also be arranged in a U-shaped section of the cleaning device, which is placed over the teeth. The brushes can be driven to clean the teeth through vibration, rotation, or other translational and/or rotational movement.

In principle, any cleaning device can be used in this method, wherein the cleaning instructions must then be adjusted accordingly. In the case of an electric toothbrush, for example, the cleaning instructions can specify the vibration speed and/or the vibration amplitude.

It is also conceivable, however, for a simple manual toothbrush to be used. The cleaning instructions then include instructions for using the manual toothbrush, which are displayed to the user, for example as corresponding notifications on a smartphone such as “Clean the lower back molars more intensively!” or “Brush the upper left canine for 10 seconds!”.

The term “tooth” refers foremost to a natural tooth of the dentition, but the cleaning device also cleans artificial teeth or artificial tooth surfaces that are present as prostheses instead of natural teeth or tooth surfaces. Teeth, including artificial teeth, are located in the user's oral cavity.

Here and in the following, it is assumed that there are 32 teeth. The procedure described here also works with a different number of teeth. There can be missing teeth, especially the wisdom teeth, or there may be more teeth in the case of hyperdontia.

The models are data models that describe processed data in connection with tooth cleaning and also define the relationships among the data. The dental model, which describes the tooth structure of a user, is an assignment of a user's teeth to corresponding data. The dental model defines a spatial arrangement of the teeth. In the simplest model, this can be the numbers 1-32. The FDI dental notation system is preferably selected.

The cleaning model is based on the dental model and uses the dental model to describe the plaque conditions of the teeth.

This can be done, for example, by associating plaque to a specific tooth in such a way that the area of the tooth surface that is covered with a biofilm or plaque is specified as a percentage.

It is also conceivable, however, for the plaque to be indicated in the form of a plaque map of the tooth, wherein the plaque map represents the surface of the assigned tooth and this map indicates where a biofilm or plaque is present on the tooth surface.

Cleaning instructions for the cleaning process are established based on the cleaning model in a predetermined manner. For example, such an assignment can be made in such a way that if 75% of a tooth is covered with a biofilm, then this tooth is cleaned for a certain duration with a certain intensity.

The expected cleaning model is preferably calculated before the cleaning process, but can also be calculated independently of the cleaning process. Although the expected cleaning model can be output to a user, it is particularly important to check how well the prediction and the actual usage match in comparison to the actual cleaning model.

Such a deviation can be caused, for example, by incorrect usage by a user.

The cleaning deviation can also be caused by a tooth misalignment that deviates from the norm. The control loop is executed as described above in order to take such a tooth misalignment into account during the cleaning.

For example, if a tooth is slightly more indented than the neighboring teeth, cleaning elements cannot reach the corresponding surface of the tooth as easily. In this case, a correspondingly longer duration or stronger cleaning intensity of the cleaning elements of the affected tooth would have to be set even though the plaque on the tooth may not be thicker than the plaque on the surrounding teeth.

Since there are usually several cleaning processes before a new determination of the cleaning model, individual deviations can add up over the individual cleaning processes so that the cleaning deviation after a first cleaning process is negligible, but after 14 cleaning processes it has a significant effect on the cleaning quality.

By selecting one or more specific cleaning processes, a certain cleaning effect is expected. By calculating an expected cleaning model and comparing it to the actual cleaning model, it is possible to assess the efficiency of one or more cleaning processes. This makes it possible to determine whether the selection of one or more cleaning processes was successful. Since the cleaning instructions are adjusted based on the cleaning deviation between the actual cleaning model and the expected cleaning model, the efficiency of the one or more cleaning processes carried out is taken into account in future cleaning process(es) and an optimized selection is made. Such an efficiency test of the individual cleaning processes is not known from the prior art explained at the beginning.

Preferably, a detection device is inserted into the oral cavity and detects tooth attitudes, tooth positions, and/or occurrences of plaque such as a biofilm.

This allows a dental model and/or a cleaning model to be generated according to the data measured by the detection device.

In various embodiments, the adjustment of the cleaning instructions through several cleaning deviations is performed in accordance with one of the following rules:
the areas of the teeth where insufficient cleaning has been detected are cleaned more intensively, e.g. by applying more pressure, or for longer,
the direction of the nozzles of the cleaning device are adjusted in such a way that the areas of the teeth in which insufficient cleaning has been detected are more in the focus of the cleaning jet, and/or
the cleaning fluid is adjusted in such a way that in the event of excessive plaque, a cleaning fluid with more particles should be used, and/or
if inflammation is detected, the cleaning fluid is adjusted to contain anti-inflammatory agents.

Such an adjustment increases the success of the cleaning process without the user having to actively set it.

Preferably, a cleaning trend is generated from a plurality of cleaning deviations in a trend module and a user receives instructions according to the cleaning trend.

Furthermore, the method can be embodied such that the cleaning model is executed by a setting module, the expected cleaning model is executed by an analysis module, the actual cleaning model is executed by an evaluation module, and the cleaning deviation is executed by a feedback module, and such that these modules and the trend module constitute respective software modules that can be executed on a computing system, which has a processor and a memory.

Since the individual models are composed of modules, some of which are different, and these individual modules interact with one another, these modules can be arranged as required. For example, the modules can be embodied on a common computing system or on several individual computing systems, wherein the models are then exchanged between the modules.

In some embodiments, the setting module, the evaluation module, the analysis module, the feedback module, and the trend module can each be executed on at least one of the following devices: the detection device, the cleaning device, a base station, a mobile terminal such as a smartphone, and/or on a central server.

The device on which at least one module is executed has a computing system.

Such a computing system can, for example, be embodied as a microprocessor, a small computer or microcomputer, a central server, a mobile computing unit (smartphone, laptop), or a PC.

According to a modification, the frequency of tooth cleaning processes is taken into account.

For example, a distinction must be drawn as to whether a user only cleans his teeth with the cleaning device once a week or whether a user cleans his teeth with it three times a day. It may also be necessary to consider whether a user cleans his teeth with other cleaning devices, such as a conventional toothbrush, in between.

In the case of product liability claims, however, the frequency of use can also be used to prove a lack of regularity of use.

Preferably, the number of cleaning processes is taken into account when generating the expected cleaning model in order to compare it to the actual cleaning model.

Alternatively, an expected cleaning model is generated for each cleaning process.

A distinction must be drawn as to whether a user only generates a new cleaning model every 2 weeks and cleans his teeth twice a day or whether a user generates a cleaning model once a day. The less frequently a user generates a cleaning model, the greater the cleaning deviation will be.

A cleaning system for cleaning teeth by removing plaque comprises a cleaning device and a detection device and in particular, is embodied to carry out a method described above.

Preferably, the detection device has a detection capsule that contains a detection fluid and the cleaning device contains a cleaning fluid.

The detection fluid contains a substance that adheres particularly well to a biofilm and when illuminated with a specific wavelength, glows with a second wavelength.

The cleaning fluid contains articles that can remove a biofilm.

Preferably, there are different cleaning capsules with different respective cleaning fluids, e.g. with different concentrations of particles and/or anti-inflammatory agents.

The selection of a specific cleaning capsule can be time-controlled, e.g. that the first ten applications use a cleaning capsule of type 1 (intensive cleaning) and then a cleaning capsule of type 2 (standard) is used for the next applications.

The selection of cleaning capsules can also be controlled by means of corresponding cleaning instructions. Depending on the requirements, a specific cleaning capsule is required for a specific cleaning fluid.

Alternatively, the cleaning fluid and/or the detection fluid can also be manually introduced into the oral cavity. For example, a predetermined amount of cleaning fluid and/or detection fluid is taken into the oral cavity by the user without swallowing and distributed in the oral cavity by mouth movements.

According to a modification, the detection capsule and/or the cleaning capsule is provided with a marker, wherein the marker contains one or more pieces of information.

This data can preferably also be added subsequently, e.g. in the distribution channel or by the user himself.

The marker contains information such as:
particle content, for example to select the cleaning program,
flavor, for example to adapt the display color to the flavor, e.g. red for cherry, blue for peppermint,
fluid composition, for example, an indication that the device must be shaken before use if the composition is critical for sedimentation,
expiration date,
cleaning fluid and/or detection fluid, for example so that they do not get mixed up,
whether it contains a disinfecting fluid,
a serial number so that, for example, capsules that have already been used can no longer be reused or refilled capsules can be detected. With multiple use or detection of imitators, also the compatibility, for example whether the capsule fits into the device,
source and distribution channel of the capsule for traceability from production through the distribution channel to the customer,
lead time from sale to use,
time in the cooling/reheating/storage station, which enables the applied reheating time to be tracked,
information about the buyer (whether the buyer is the user or someone else is using the device),
name of the user for a personal greeting,
language of the user,
home address including country and time zone of the user,
email address of the user,
telephone number of the user,
preference for the display background (images, color gradients),
user competence in beginner/advanced/expert for user-friendliness and expert setting options,
calibration data for gestures from previous device.

In some embodiments, the marker is an RFID tag, in particular an NFC tag.

Another option is for the marker to have a barcode, QR code, color code, or other printed marker.

Preferably, the detection device and/or the cleaning device also has a marker, in particular an NFC tag, as described above.

This marker can include one or more of the following pieces of information:
name of the user, e.g. for a personal greeting,
language of the user,
home address including country and time zone of the user,
email address of the user,
telephone number of the user,
preference for the display background (images, color gradients),
user competence in beginner/advanced/expert for user-friendliness and expert setting options, and/or
calibration data for gestures from previous device.

The invention is explained in more detail below with reference to the drawing. In the drawings:

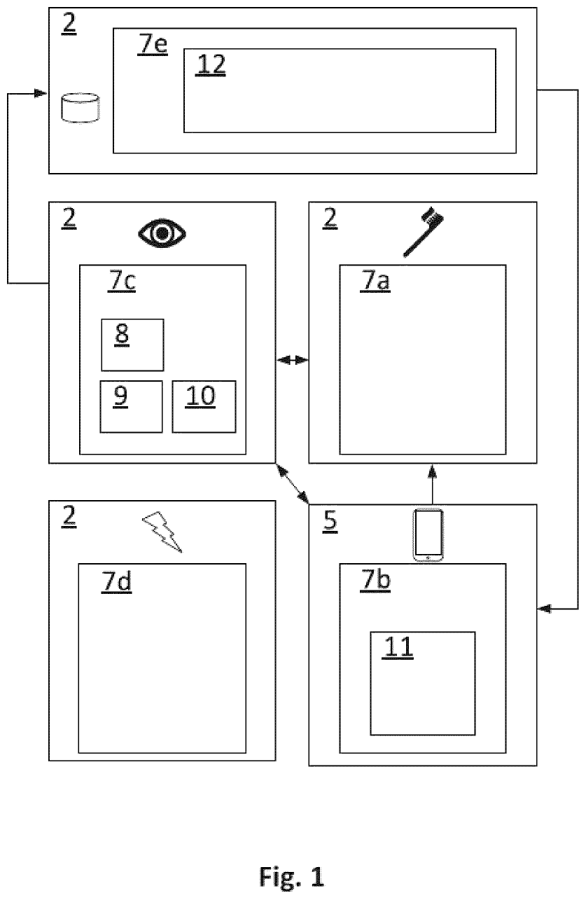

FIG. 1 shows a schematic block diagram of the cleaning system,

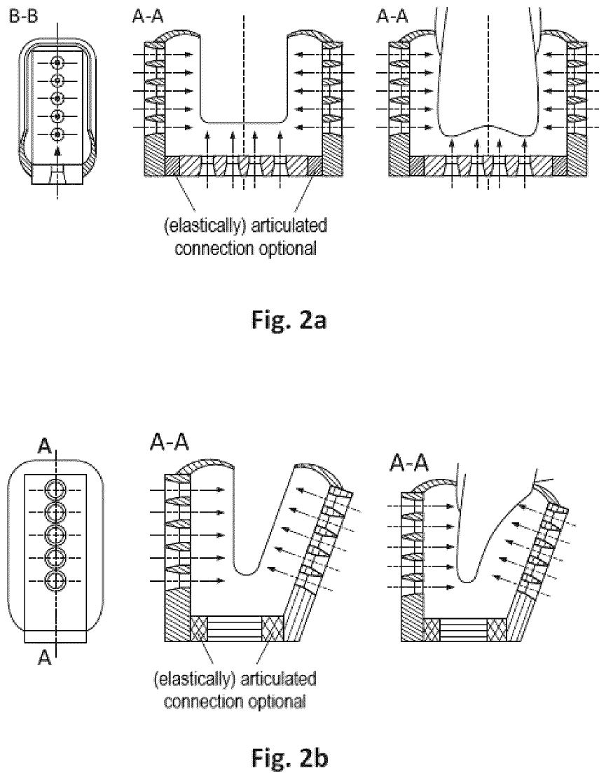

FIG. 2a schematically depicts a part of the cleaning device for cleaning molars,

FIG. 2b schematically depicts a part of the cleaning device for cleaning incisors,

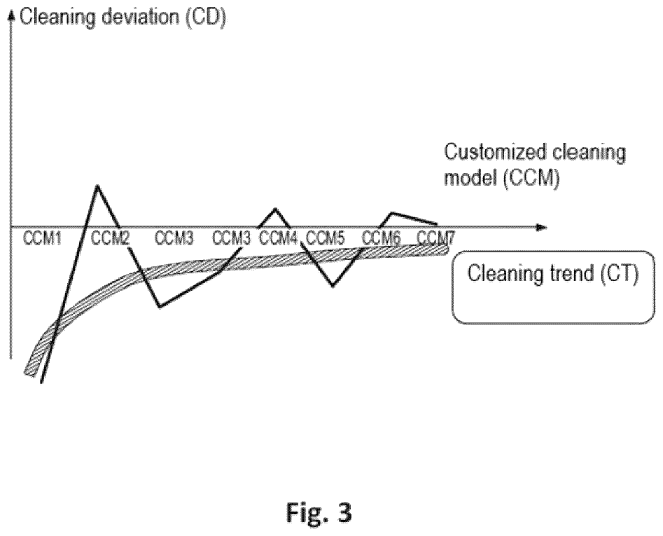

FIG. 3 shows the curve of a cleaning trend over time, and

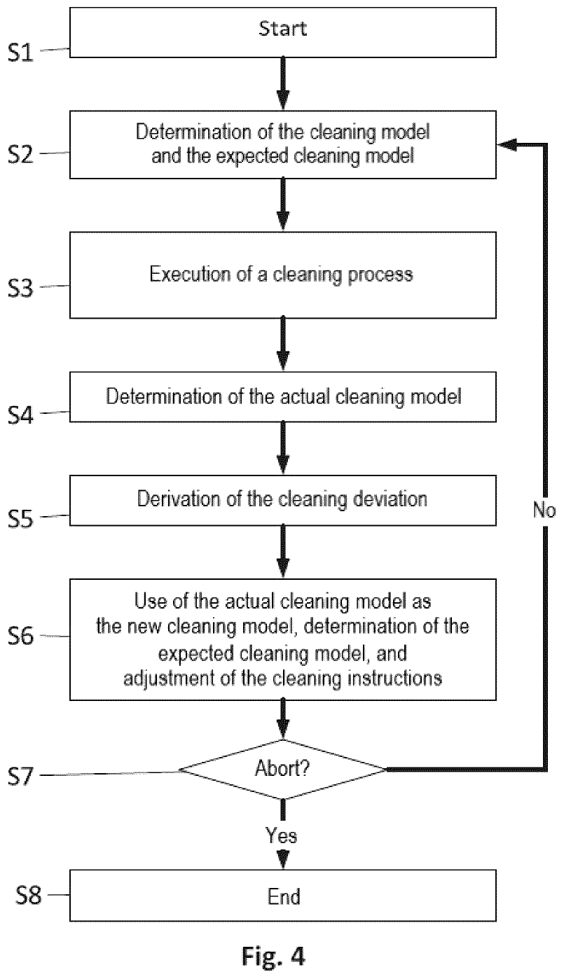

FIG. 4 shows a flowchart of the cleaning method.

The cleaning system 1 has a cleaning device 2, a detection device 3, a base station 4, an interface for a data terminal 5 such as a smartphone 5, and a central server 6, each of which has a computing system 7. Each computing system 7 in turn comprises a processor and a memory.

Such a detection device 3 is described in detail, for example, in the not yet published German patent application DE 10 2022 102 045.2 and reference is made to this patent application in its entirety. The detection device 3 is embodied to generate the cleaning model 14.

In principle, the detection device 3 is embodied in such a way that a U-shaped section of the detection device 3 is inserted into a user's oral cavity. The U-shaped section of the detection device 3 has a sensor array.

The U-shaped section is placed onto the teeth so that all tooth surfaces can be detected by a sensor.

To make a biofilm more visible, a detection fluid is introduced into the oral cavity before the detection process. The detection fluid interacts with the biofilm and, under the influence of a light with a predetermined wavelength, causes the biofilm to glow at a different predetermined wavelength.

The detection fluid is placed in a detection capsule that can be inserted into the detection device 3. The detection device then withdraws the detection fluid and pumps it onto the teeth.

For this purpose, the detection device 3 consists of a handpiece, which can have a display on the side facing away from the user. On the side facing the user, there is a mouthpiece that is inserted into the oral cavity and guides the sensor unit over the teeth.

The mouthpiece has at least one camera unit with a camera.

The camera unit alone, for example, has the dimensions 1×1×2.7 mm and the entire sensor unit, including a protective glass, PCB (printed circuit board), filter, and camera holder, has a diameter of 8 mm and a height of 3.8 mm. With these dimensions, the unit can be easily inserted into an oral cavity.

To ensure a good signal-to-noise ratio, an optical long-pass filter is preferably placed directly in front of the camera, which filter ideally has a cut-off wavelength of around 510 nm and cuts off signals below this. In addition, a band-pass or short-pass filter can be placed in front of the LEDs that illuminate the area to be detected in order to limit the wavelength spectrum of the LEDs.

The cleaning device 2 is described in detail in the not yet published German patent application 10 2020 134 154.7 and reference is made to this patent application in its entirety.

In principle, the cleaning device 2 is embodied in such a way that a U-shaped section of the cleaning device 2 is inserted into the oral cavity of a user. The U-shaped section of the cleaning device 2 has a nozzle arrangement and is placed over the teeth. A nozzle is positioned over each tooth surface.

The nozzle of the nozzle arrangement pumps a cleaning fluid onto the teeth in such a way that a biofilm on the teeth is removed.

In this exemplary embodiment, the cleaning fluid is a cleaning fluid and can contain particles in order to accelerate the removal process.

The cleaning fluid is contained in a cleaning capsule that can be inserted into the cleaning device. The cleaning device 2 then withdraws the cleaning fluid and pumps it onto the teeth.

To prevent a cleaning fluid from entering the oral cavity in an uncontrolled manner, sealing elements in the form of sealing lips are provided on a nozzle arrangement, i.e. a sequence of nozzles. The sealing lips seal off the area around the nozzle arrangement so that a fluid cushion like an air cushion is produced, but with the difference that fluid should not escape to the outside.

Accordingly, a nozzle housing is depicted very schematically in this simplified embodiment (FIGS. 2a and 2b), which has a nozzle opening, wherein the nozzle housing is embodied in a simplified way here with a rectangular cross-section.

Corresponding sealing elements are provided on an outer surface of the nozzle housing on the outlet side facing the surface to be cleaned, in the region of the outer edges. The sealing elements can also be embodied as a single circumferential sealing element and are rubber-elastic so that they adapt to the geometry of a surface to be cleaned and are also able to ensure a certain internal pressure of the fluid in the enclosed space.

In particular, the sealing elements can be embodied as circumferential sealing elements or circumferential sealing lips that extend away from the surface on the outlet side of the nozzle housing. The sealing elements or sealing lips in this case can be embodied with a certain inherent rigidity so that the basic shape of the enclosed space is formed and retained.

In addition, the sealing elements or sealing lips can be embodied as hollow in order to be inflated with the cleaning fluid, for example, so that the sealing elements only achieve the shape that they are supposed to have during operation after they have been inflated, for example with the cleaning fluid or other fluids that are supplied separately.

In a simple embodiment, in particular for cleaning in the region of the molars, the nozzle housing has, for example, five nozzle openings positioned one above the other in the (jaw) sides or opposite the tooth flanks and four nozzle openings positioned next to one another at the base, opposite a tooth crown, and the corresponding sealing elements, which seal off the corresponding enclosed space.

In the incisor region, such an embodiment can in particular differ in the shape of the sealing elements, but also in the angled arrangement of the nozzle housing and the base nozzle housing in order to achieve an adaptation to the incisor region. The base nozzle housing can also be embodied without nozzles in the incisor region if the nozzle openings in the nozzle housings that are positioned opposite each other ensure sufficient flow to an incisor.

In order to prevent the enclosed space from filling up with fluid without discharging this fluid again in a controlled manner, in one embodiment, a reverse suction according to the invention is provided. In the simplest case, the fluid is discharged through the nozzle or nozzle opening into the enclosed space and then sucked back through the nozzle opening again. In order to ensure that the enclosed space does not have to be filled solely by one or more nozzles before the actual cleaning operation begins, an inlet can be provided for each one of one or more or all of the nozzles.

Since the cleaning fluid can in particular contain particles, the cleaning performance is also determined by the particles and the particle flow. Since the particles have a higher density than the surrounding fluid, the fluid moves tangentially away from the surface when it strikes the surface, but the particles strike the surface and damage the biofilm or plaque, thereby contributing to its removal.

The cleaning capsule and the detection capsule each have an NFC tag that contains several pieces of information. The NFC tag can be read by the individual devices. The stored information includes, among other things:
type of fluid contained,
flavor of the fluid,
expiration date, and
serial number.

Various executable software modules are stored on the computer systems 7 of the individual devices. A setting module 8, an evaluation module 9, and an analysis module 10 are provided in the detection device 3, a feedback module 11 is provided in the smartphone 5, and a trend module 12 is provided in the central server 6.

The modules are embodied in such a way that they can communicate with one another via wireless communication such as WLAN, Bluetooth, 433 MHz band, Zigbee, and Z-Wave.

In this exemplary embodiment, a cleaning model 14 is constituted by the setting module 8, an expected cleaning model 15 is constituted by the analysis module 10, an actual cleaning model 16 is constituted by the evaluation module 9, a cleaning deviation 17 is constituted by the feedback module 11, and a cleaning trend 18 is constituted by the trend module 12.

In principle, the individual modules can also be executed on other devices. For example, it is conceivable for the base station 4 to execute the feedback module 11 instead of the smartphone 5. The base station 4 can also be embodied in such a way that several or all of the modules can be executed on it. The detection device 3 and the cleaning device 2 then send and receive the data accordingly.

The dental model 13 used here corresponds to the FDI (Fédération Dentaire Internationale) dental notation system. In it, the teeth are divided into four quadrants, with quadrant 1 being the upper jaw on the right, quadrant 2 being the upper jaw on the left, quadrant 3 being the lower jaw on the left, and quadrant 4 being the lower jaw on the right. Each quadrant indicates the tens digit of a tooth number for the dental model 13. For example, the teeth in quadrant 1 are teeth 11-18 and the teeth in quadrant 4 are teeth 41-48. The ones digits are determined by the position of the teeth in the respective quadrant. The first two incisors are numbered 1 and 2, the canine tooth is numbered 3, the premolars are numbered 4 and 5, the molars are numbered 6 and 7 and the wisdom tooth is numbered 8. For example, the canine tooth of the upper jaw on the left is numbered 23.

In this exemplary embodiment, the cleaning model 14 is an addition to the dental model 13 with a plaque value that represents the plaque coverage of a tooth as a percentage of the total surface area of the respective tooth. For example, the plaque value is 50% if half of the surface area of the tooth is covered with a biofilm. The cleaning model 14 has a percentage value like this for each entry of each tooth from the dental model 13. For example, if the wisdom tooth of the lower jaw on the right is 32% covered with a biofilm, the entry would be: “48:32%”.

This plaque value is preferably determined for each tooth by the detection device 3 and is assigned to the cleaning model 14. In principle, it is also possible to determine plaque values for several neighboring teeth.

It is also conceivable for several plaque values to be determined for a tooth in order to take into account the inner, outer, and optionally occlusal surfaces of the tooth.

The cleaning device 2 with the computing system 7a receives the user's cleaning model 14 and generates cleaning instructions based on this cleaning model 14.

These cleaning instructions are instructions as to how long and with what intensity the nozzles should act on the teeth. Basically, the more severe the plaque, which is determined by the plaque value, the longer and more intensive the tooth cleaning is, which is accomplished by increasing the cleaning duration and/or the pumping power. The relationship between the cleaning duration and/or pumping power and the plaque value can be linear, exponential, or quadratic.

The cleaning instructions for the respective plaque values can be stored in a database or table or can be provided by a function.

For example, if an entry of the cleaning model 14 is “14:73%”, this means that the front premolar of the upper jaw on the right is covered with 73% biofilm. The cleaning device 2 compares this to a stored table and determines that if the anterior premolar of the upper jaw on the right is between 70 and 80% covered with biofilm, then the corresponding nozzle operates at 90% of the maximum intensity for a period of 1 to 2 seconds.

The user's teeth are then cleaned in one cleaning process using all of the cleaning instructions.

Based on the cleaning instructions sent by the cleaning device 2, the detection device 3 on which an analysis module 10 is executed calculates an expected cleaning model 15, which in this exemplary embodiment has the same formatting as the cleaning model 14. This means that for a single tooth with the entry “14:73%” of the cleaning model 14, a corresponding expected cleaning model 15 would look like this: “14:3%”. In other words, the detection device calculates that the plaque coverage will fall from 73% to 3%.

The detection device 3 also determines the actual cleaning model 16 after a cleaning process. The actual cleaning model 16 is determined using the same method as the cleaning model 14. The data structure of the cleaning model 16 is also the same as that of the cleaning model 14. The smartphone 5, which has the computing system 7b on which the feedback module 11 is executed, receives the expected cleaning model 15 and the actual cleaning model 16 from the detection device 3 and compares them in order to determine the cleaning deviation 17. For example, if the actual cleaning model 16 has been determined in such a way that the entry for a tooth is “14:13%”, then a cleaning deviation 17 will have the following form “14:10%”, since the difference between the expected value and the target value for tooth 14 is 10%.

The actual cleaning model 16 and the cleaning model 14 are transmitted from the detection device 3 to the central server 6, which is executed by the computing system 7c there, on which the trend module 12 is executed, so that a cleaning trend 18 is determined. The cleaning trend 18 is sent from the central server 6 to the smartphone 5, where a user can be notified of a potential error, for example if the cleaning trend indicates that the user is not using the cleaning device 2 correctly. It is also possible for the cleaning trend 18 to adjust the cleaning instructions of a cleaning device 2.

The procedure for cleaning teeth with a tooth cleaning device is described below (see FIG. 4).

The procedure begins with step S1.

In the next step (S2), the cleaning model 14 and the expected cleaning model 15 are determined.

For this purpose, the detection device 3 is inserted into a user's oral cavity and the detection device 3 detects the amount of plaque on the user's individual teeth.

The setting module 8 of the detection device 3 generates the cleaning model 14 by adding a predetermined dental model 13 with the results of the detection.

The appearance of the dental model 13 and the cleaning model 14 is described in detail above.

The cleaning model 14 is sent to the cleaning device 2. The cleaning device 2 uses the cleaning model 14 to generate cleaning instructions and sends the cleaning instructions back to the detection device 3.

The detection device 3 generates an expected cleaning model 15 based on the cleaning model 14 and the cleaning instructions.

In the simplest case, it is possible to read from a stored table what the expected cleaning model 15 will be for a given cleaning model 14 with given cleaning instructions. As described above, an entry of the cleaning model 14 is “14:73%”, for example. This means that the anterior premolar of the upper jaw on the right is 73% covered with biofilm. The cleaning device 2 compares this to a stored table and determines that if the anterior premolar of the upper jaw on the right is between 70 and 80% covered with biofilm, then the corresponding nozzle must operate at 90% of the maximum intensity for a period of 1.5 seconds.

A cleaning process is then carried out (step S3).

For this purpose, the cleaning device 2 is inserted into the user's oral cavity and the nozzles clean the teeth in accordance with the cleaning instructions.

This is followed by step S4 in which the actual cleaning model 16 is determined.

Like the cleaning model 14, the actual cleaning model 16 is determined by the detection device 3. The actual cleaning model 16 can be determined in exactly the same way as the cleaning model 14. The data structure of the cleaning model 16 is also the same as that of the cleaning model 14.

In the next step (S5), a cleaning deviation 17 is derived. For this purpose, the detection device 3 sends the expected cleaning model 15 and the actual cleaning model 16 to the feedback module 11 of the smartphone 5.

The feedback module 11 checks how much the actual cleaning model 16 and the expected cleaning model 15 differ from each other. In the examples described above for the actual cleaning model 16 and the expected cleaning model 15, the difference is 10% (see above).

This 10% corresponds to the cleaning deviation value for the corresponding tooth. The cleaning deviation 17 has the same data structure as the cleaning model 14, the expected cleaning model 15, and the actual cleaning model 16. The percentage in this case, however, indicates the difference between the expected cleaning model 15 and the actual cleaning model 16.

The cleaning deviation value can also be negative if the cleaning is better than predicted.

Then, in step S6, the actual cleaning model 16 is used as the new cleaning model 14, a new expected cleaning model 15 is determined (comparable to step S2) and the cleaning instructions are adjusted.

The cleaning instructions, however, are preferably adjusted only if the cleaning deviation 17 is above a predetermined threshold value. This can be 5%, for example.

It is also conceivable to select only the amount of the cleaning deviation 17 above a predetermined threshold value as a criterion for adjusting the cleaning instructions. For example, the cleaning deviation 17 can also be negative if the teeth are cleaned better than calculated. Even then, it makes sense to adjust the cleaning instructions since it may be possible to save time and apply less pressure to the teeth and gums through the nozzles, thus protecting the user's gums.

A corresponding instruction is sent from the feedback module 11 of the smartphone 5 to the cleaning device 2 so that the cleaning instructions can be adjusted.

The new cleaning model 14 is in turn sent to the detection device 3, which receives the revised cleaning instructions from the cleaning device 2.

In the subsequent step S7, a check is run as to whether a termination condition exists.

A termination condition exists, for example, if the user has disposed of the cleaning system 1 or has not used it for a predetermined period of time, such as one year. It is also conceivable that such a termination condition exists if the user of the cleaning system has changed, for example because the appliance has been resold.

If there is no abort condition, then the procedure is repeated and starts with step S2.

If a termination condition does exist, then the procedure ends with step S8.

Another option is to determine the cleaning trend 18 with the trend module 12 on the central server 6. In this case, each cleaning model 14 and each actual cleaning model 16 is sent from the detection device 3 to the central server 6. The trend module 12 of the central server 6 determines the cleaning trend 18 from the individual cleaning models 14 and actual cleaning models 16 by checking the degree of cleaning for the individual teeth over time.

With normal use, it can be assumed that the overall cleaning success compared to the condition before the first use will increase over time with regular use of the cleaning device 2, i.e. the user's teeth will gradually be freed of the biofilm and the teeth will become cleaner.

It is possible, however, that individual teeth or rows of teeth or even all of the user's teeth may not become cleaner or may even become more covered with plaque. This is a strong indication that the user is using the cleaning device 2 incorrectly. For example, the user may not be placing the cleaning device 2 completely over his teeth.

If the cleaning trend 18 does not have the desired curve, then this could also be an indication that the cleaning device 2 is defective and/or that individual components need to be replaced because they have become defective or worn out after intensive use.

The central server 6 then sends a corresponding notification to the smartphone 5 to inform the user. In this case, the user can be prompted to use the cleaning device 2 correctly in accordance with the instructions or, if need be, to replace individual elements.

The cleaning trend 18 can also provide information on how to adjust the cleaning instructions In this case, a user is not necessarily notified by the smartphone 5; instead, the central server 6 sends corresponding instructions to the smartphone 5, which forwards them to the cleaning device 2 in order to adjust the cleaning instructions.

Preferably, in the steps before the generation of the cleaning model 14 and/or the actual cleaning model 16, the NFC tag of the detection capsule is read out by the detection device 3 so that the information stored there is stored as meta data in the corresponding model.

It is also advantageous if the NFC tag of the cleaning capsule is read by the cleaning device 2 before the cleaning process and sent to the detection device 3. Alternatively, the data can also be sent to the smartphone 5 for further processing.

Another option is for the cleaning device 2 and/or the detection device 3 to have sensors that allow conclusions to be drawn about the condition of these devices. For example, the sensors can be acceleration sensors that measure acceleration values due to device vibrations and analyze the change in them over time. The data can be sent to the central server 6 in order for the measured data to be analyzed there. In the case of acceleration sensors, for example, it can be determined that the frequency spectrum and/or the amplitude of the acceleration is changing. The central server 6 can then send a corresponding message to the smartphone 15 to inform the user of the necessary steps.

Acceleration sensors can also be used to detect whether the device has suddenly stopped measuring gravitational acceleration and is therefore in free fall. Based on the length of this phase, the drop height can be determined and it is thus possible to reject unjustified warranty claims or to send a message to the user that the device needs to be serviced.

In an alternative embodiment, the images captured by the detection device 3 are analyzed using a self-learning system.

Because of the detection fluid used and the selected wavelength filter, the contrast for the biofilm is very high. The biofilm has a high signal-to-noise ratio, but this means that other objects such as the tooth surface are difficult to detect. The areas that are not covered with a biofilm are difficult to distinguish from one another. The main difficulty here is to distinguish the tooth from the surrounding tissue such as the gums, lips, and other features in the mouth.

With the help of artificial intelligence, pattern recognition can analyze slight differences in brightness to distinguish a tooth from non-tooth objects.

Preferably, trained pattern recognition is used here.

For this purpose, captured images are analyzed manually by manually marking the area of the image that is to be assigned to the tooth. This, together with other information such as the camera position and/or tooth position is input into the self-learning system, which preferably comprises a neural network, for learning purposes.

If the self-learning system has been supplied enough images with corresponding additional data and markings for learning, then the system is able to mark new images of teeth independently in such a way that the tooth areas can be distinguished from non-tooth areas.

According to a modification, one or more devices of the cleaning system can be unlocked by means of a biometric procedure.

### REFERENCE NUMERALS

1 cleaning system
2 cleaning device
3 detection device
4 base station
5 smartphone
6 central server
7 computing system
8 setting module
9 evaluation module
10 analysis module
11 feedback module
12 trend module
13 dental model
14 cleaning model
15 expected cleaning model
16 actual cleaning model
17 cleaning deviation
18 cleaning trend

1. A method for cleaning teeth with a tooth cleaning device for removing plaque comprising using the following models:
a dental model that describes the tooth structure of a user,
a cleaning model of the respective user, which describes the dental model with a plaque condition, wherein the cleaning model is used as a basis for establishing cleaning instructions for a tooth cleaning process,
an expected cleaning model, which describes the dental model with an expected cleaning state that should be achieved after performing a specific cleaning procedure as defined by the cleaning model,
an actual cleaning model that is actually achieved after performing the particular cleaning operation as defined by the cleaning model, wherein the actual cleaning model is measured with corresponding sensors,
a cleaning deviation, which is determined by comparing the expected cleaning model to the actual cleaning model, wherein a control loop with the following steps is executed:
a) determining the cleaning model and the expectation-cleaning model,
b) executing a cleaning process as defined by the cleaning model,
c) determining the actual cleaning model,
d) deriving the cleaning deviation from the actual cleaning model and the expected cleaning model,
e) using the actual cleaning model as the new cleaning model, determining the expected cleaning model based on the new cleaning model, and adjusting the cleaning rules based on the cleaning deviation,
f) repeating steps b) to e) until a predetermined termination criterion is reached.

2. The method according to claim 1,
characterized in that
a detection device is inserted into the oral cavity and detects tooth attitudes, tooth positions, and/or occurrences of plaque such as a biofilm.

3. The method according to claim 1,
characterized in that
the adjustment of the cleaning instructions through several cleaning deviations is performed in accordance with one of the following rules:
the areas of the teeth where insufficient cleaning has been detected are cleaned more intensively, e.g. by applying more pressure, or for longer,
the direction of the nozzles of the cleaning device are adjusted in such a way that the areas of the teeth in which insufficient cleaning has been detected are more in the focus of the cleaning jet, and/or
the cleaning fluid is adjusted in such a way that in the event of excessive plaque, a cleaning fluid with more particles should be used, and/or,
if inflammation is detected, the cleaning fluid is adjusted to contain anti-inflammatory agents.

4. The method according to claim 1,
characterized in that
a cleaning trend (CT) is generated from a plurality of cleaning deviations (CD) in a trend module and a user receives instructions according to the cleaning trend.

5. The method according to claim 1,
characterized in that
the cleaning model is executed by a setting module, the expected cleaning model is executed by an analysis module, the actual cleaning model is executed by an evaluation module, and the cleaning deviation is executed by a feedback module, and in that these modules and the trend module constitute respective software modules that can be executed on at least one computing system, which has a processor and a memory.

6. The method according to claim 5,
characterized in that
the setting module, the evaluation module, the analysis module, the feedback module, and the trend module can each be executed on at least one of the following devices: the detection device, the cleaning device, a base station, a data terminal such as a smartphone, and/or on a central server.

7. The method according to claim 6,
characterized in that
the setting module is executed on the detection device, the evaluation module is executed on the detection device, the analysis module is executed on the detection device, the feedback module is executed on the smartphone, and the trend module is executed on the central server.

8. The method according to claim 1,
characterized in that
the frequency of tooth cleaning processes is taken into account.

9. The method according to claim 1,
characterized in that
the number of cleaning processes is taken into account when generating the expected cleaning model in order to compare it to the actual cleaning model.

10. A cleaning system for cleaning teeth by removing plaque comprises a cleaning device and a detection device and in particular, is embodied to carry out a method according to claim 1.

11. The cleaning system according to claim 10,
characterized in that
the detection device has a detection capsule that contains a detection fluid and the cleaning device has a cleaning capsule that contains a cleaning fluid.

12. The cleaning system according to claim 10,
characterized in that
the detection capsule and/or the cleaning capsule is provided with a marker, wherein the marker contains one or more pieces of information.

13. The cleaning system according to claim 12,
characterized in that
the marker is an RFID tag, in particular an NFC tag.
